SEQUENCE LISTING (11 sequences)

SEQ ID NO: 1 - DNA, 29 bp, synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, synthetic construct
  source 1..29: mol_type = other DNA
  modified_base 23: mod_base = i
aatgatacggcgaccaccgaganctacac

SEQ ID NO: 3 - DNA, 29 bp, synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 7 - DNA, 29 bp, synthetic construct
  source 1..29: mol_type = other DNA
  modified_base 23: mod_base = OTHER; note = allyl-T
aatgatacggcgaccaccgaganctacac

SEQ ID NO: 8 - DNA, 35 bp, synthetic construct
  source 1..35: mol_type = other DNA
gctggcacgtccgaacgcttcgttaatccgttgag

SEQ ID NO: 9 - DNA, 35 bp, synthetic construct
  source 1..35: mol_type = other DNA
cgtcgtctgccatggcgcttcggtggatatgaact

SEQ ID NO: 10 - DNA, 35 bp, synthetic construct
  source 1..35: mol_type = other DNA
acggccgctaatatcaacgcgtcgaatccgcaact

SEQ ID NO: 11 - DNA, 34 bp, synthetic construct
  source 1..34: mol_type = other DNA
gccgcgttacgttagccggactattcgatgcagc

(19) United States
(12) Patent Application Publication
(10) Pub. No.: US 2024/0301487 A1
(43) Pub. Date: Sep. 12, 2024

(54) BIOMOLECULE IMMOBILIZATION METHOD

(71) Applicant: ILLUMINA, INC., San Diego, CA (US)

(72) Inventors: Samantha Kelly Brittelle, San Diego, CA (US); Tyler J. Dill, San Diego, CA (US); Michelle Kate Fu, San Diego, CA (US); Ryan David McCurdy, San Diego, CA (US); Michael L. Neville, San Diego, CA (US); Sean M. Ramirez, San Diego, CA (US); Stuart A. Sardo, San Diego, CA (US)

(21) Appl. No.: 18/600,605

(22) Filed: Mar. 8, 2024

Related U.S. Application Data
(60) Provisional application No. 63/489,380, filed on Mar. 9, 2023.

(51) Int. Cl. C12Q 1/6874; C12Q 1/6806

(52) U.S. Cl. CPC C12Q 1/6874; C12Q 1/6806

(57) ABSTRACT

In an example method, a grafting solution is applied to a patterned substrate using a liquid-phase thin-film deposition technique. The patterned substrate includes a lane surrounded by, or a plurality of depressions separated by interstitial regions; and a polymer in the lane or in each of the plurality of depressions. The polymer is functionalized with a first click reaction moiety. The grafting solution includes a solvent; a polymer matrix material dissolved in the solvent; and primers of a primer set dissolved in the solvent, each of the primers being terminated with a second click reaction moiety. The applied grafting solution is dried. During drying, a solid polymer matrix is formed and at least some of the primers attach to the polymer i) via the first and second click reaction moieties and ii) in at least a portion of the lane or in at least some of the plurality of depressions.

### CROSS-REFERENCE TO RELATED APPLICATIONS

This application claims the benefit of U.S. Provisional Application Ser. No. 63/489,380, filed Mar. 9, 2023, the content of which is incorporated by reference herein in its entirety.

### REFERENCE TO SEQUENCE LISTING

The Sequence Listing submitted herewith is hereby incorporated by reference in its entirety. The name of the file is ILI254B_IP-2497-US_Sequence_Listing.xml, the size of the file is 14,892 bytes, and the date of creation of the file is Mar. 3, 2024.

### BACKGROUND

Some biological and/or chemical vessels, such as assay plates and flow cells, include designated reaction areas, where surface chemistry that enables a desired interaction or reaction is localized. When a reactive species is introduced into the vessel, the reactive species interacts or reacts with the surface chemistry to create a detectable signal (e.g., an electrical signal or an optical signal). Many vessels are configured with multiple reaction areas in fluid communication with a single flow channel.

### SUMMARY

Disclosed herein are methods that enable the spatially controlled attachment of a biomolecule (e.g., oligonucleotide primers of a primer set, enzymes, proteins, DNA, RNA, aptamers, or some combination of these) in a desired region of a patterned substrate. A grafting solution containing the biomolecule is capable of being deposited with high precision using a liquid-phase thin-film deposition technique. The grafting solution also contains a polymer matrix that undergoes rapid curing at relatively mild conditions to form a solid state. It has been found that the attachment of the biomolecule to the desired region occurs rapidly upon drying of the matrix rather than, e.g., when the matrix is in the liquid state. Thus, the biomolecule attachment reaction occurs simultaneously with drying. Moreover, in the examples disclosed herein, the biomolecule attachment reaction is a copper-free click reaction, and does not utilize a salt or high temperatures (e.g., from 60° C. to about 80° C.).

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of a patterned substrate or of a flow cell including the patterned substrate;

FIG. 1B is an enlarged, and partially cutaway view of an example of the architecture of the patterned substrate;

FIG. 1C is an enlarged, and partially cutaway view of another example of the architecture of the patterned substrate;

FIG. 2A through FIG. 2D together schematically depict an example of the method disclosed herein for immobilization of a biomolecule in desired regions of a patterned substrate, where FIG. 2A depicts an example of the patterned substrate including multiple channels, each of which includes depressions separated by interstitial regions, FIG. 2B depicts the deposition of a grafting solution in one channel of the patterned substrate of FIG. 2A, FIG. 2C depicts the dried grafting solution and the biomolecule attached to a polymer in each of the depressions in the one channel, and FIG. 2D depicts the patterned substrate after the cured grafting solution is removed, where the biomolecule remains attached to the polymer in each of the depressions located in the one channel;

FIG. 2A, and FIG. 2E through FIG. 2G together schematically depict an example of the method disclosed herein for immobilization of a biomolecule in a desired region of a patterned substrate, where FIG. 2A depicts an example of the patterned substrate including multiple channels, each of which includes depressions separated by interstitial regions, FIG. 2E depicts the deposition of a grafting solution in one depression of the patterned substrate of FIG. 2A, FIG. 2F depicts the cured grafting solution and the biomolecule attached to a polymer in the one depression, and FIG. 2G depicts the patterned substrate after the cured grafting solution is removed, where the biomolecule remains attached to the polymer in the one depression;

FIG. 3 depicts a cross-sectional view of one example of a flow cell including one patterned substrate;

FIG. 4 depicts a cross-sectional view of another example of a flow cell including two patterned substrates; and

FIG. 5 depicts a cross-sectional view of still another example of a flow cell including one patterned substrate integrated over a complementary metal oxide semiconductor chip.

### DEFINITIONS

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, adjacent, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

An “acrylamide monomer” is a monomer with the structure

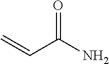

or a monomer including an acrylamide group, where each H may alternatively be an alkyl, an alkylamino, an alkylamido, an alkylthio, an aryl, a glycol, and optionally substituted variants thereof. Examples of the monomer including an acrylamide group include azido acetamido pentyl acrylamide:

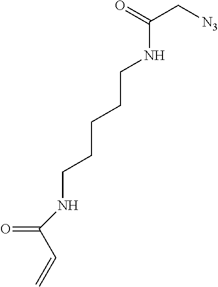 [SMILES: C=CC(=O)NCCCCCNC(=O)CN=[N+]=[N-]]

### and N-isopropylacrylamide:

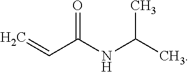

Other acrylamide monomers may be used.

The term “active area” refers to the region of a patterned substrate where a reaction can be carried out. During fabrication of the patterned substrate, the active area may include a polymer that is capable of attaching primers that can participate in nucleic acid template amplification. In the final patterned substrate, the active area may include the polymer with the primers attached thereto.

The term “activation,” as used herein, refers to a process that generates reactive groups at the surface of a substrate. Activation may be accomplished using silanization or plasma ashing. It is to be understood that activation may be performed in any of the methods disclosed herein. When activation is performed, it is to be understood that a silanized layer or —OH groups (from plasma ashing) are present to covalently attach the polymer to the underlying support or layer.

An “aldehyde,” as used herein, is an organic compound containing a functional group with the structure-CHO, which includes a carbonyl center (i.e., a carbon double-bonded to oxygen) with the carbon atom also bonded to hydrogen and an R group, such as an alkyl or other side chain. The general structure of an aldehyde is:

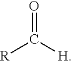

As used herein, “alkyl” refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

As used herein, “alkylamino” refers to an alkyl group in which one or more of the hydrogen atoms are replaced by an amino group, where the amino group refers to an —NRₐR_{b} group, where Rₐ and R_{b} are each independently selected from a C1-C6 alkyl, C2-C6 alkenyl, C2-C6 alkynyl, C3-C7 carbocycle, C6-C10 aryl, a 5-10 membered heteroaryl, and a 5-10 membered heterocycle.

As used herein, “alkylamido” refers to an alkyl group in which one or more of the hydrogen atoms are replaced by a C-amido group or an N-amido group. A “C-amido” group refers to a “—C(═O)N(RₐR_{b})” group in which Rₐ and R_{b} can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. An “N-amido” group refers to a “RC(═O)N(Rₐ)—” group in which R and Rₐ can independently be selected from the group consisting of alkyl, alkenyl, alkynyl, cycloalkyl, cycloalkenyl, cycloalkynyl, aryl, heteroaryl, heteroalicycle, aralkyl, or (heteroalicyclic)alkyl. Any alkylamido may be substituted or unsubstituted.

As used herein, “alkylthio” refers to RS—, in which R is an alkyl. The alkylthio can be substituted or unsubstituted.

As used herein, “alkene” or “alkenyl” or “olefin” refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

As used herein, “alkyne” or “alkynyl” refers to a straight or branched hydrocarbon chain containing one or more triple bonds. The alkynyl group may have 2 to 20 carbon atoms.

An “amino” functional group refers to an —NRₐR_{b} group, where Rₐ and R_{b} are each independently selected from hydrogen

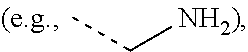

C1-6 alkyl, C2-6 alkenyl, C2-6 alkynyl, C3-7 carbocyclyl, C6-10 aryl, 5-10 membered heteroaryl, and 5-10 membered heterocyclyl, as defined herein.

The term “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl. Any aryl may be a heteroaryl, with at least one heteroatom, that is, an element other than carbon (e.g., nitrogen, oxygen, sulfur, etc.), in ring backbone.

As used herein, the term “attached” refers to the state of two things being joined, fastened, adhered, connected or bound to each other, either directly or indirectly. For example, a nucleic acid can be attached to a polymer by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

An “azide” or “azido” functional group refers to —N₃.

As used herein, a “bonding region” refers to an area of a patterned substrate that is to be bonded to another material, which may be, as examples, a spacer layer, a lid, another substrate, etc., or combinations thereof (e.g., a spacer layer and a lid, or a spacer layer and another substrate). The bond that is formed at the bonding region may be a chemical bond (as described above), or a mechanical bond (e.g., using a fastener, etc.).

As used herein, “carbocycle” means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl. Any of the carbocycles may be heterocycles, with at least one heteroatom in ring backbone.

As used herein, the term “carboxylic acid” or “carboxyl” as used herein refers to —COOH.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s). In some examples, cycloalkyl groups can contain 3 to 8 atoms in the ring(s). A cycloalkyl group may be unsubstituted or substituted. Example cycloalkyl groups include cyclopropyl, cyclobutyl, cyclopentyl, cyclohexyl, cycloheptyl, and cyclooctyl.

As used herein, “cycloalkenyl” or “cycloalkene” means a carbocycle ring or ring system having at least one double bond, wherein no ring in the ring system is aromatic. Examples include cyclohexenyl or cyclohexene and norbornenyl or norbornene.

As used herein, “cycloalkynyl” or “cycloalkyne” means a carbocycle ring or ring system having at least one triple bond, wherein no ring in the ring system is aromatic. An example is cyclooctyne. Another example is bicyclononyne. Still another example is dibenzocyclooctyne (DBCO).

As used herein, the term “depression” refers to a discrete concave feature defined in a substrate and having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As examples, the depression can be a well or two interconnected wells.

The term “each,” when used in reference to a collection of items, is intended to identify an individual item in the collection, but does not necessarily refer to every item in the collection. Exceptions can occur if explicit disclosure or context clearly dictates otherwise.

The term “epoxy” (also referred to as a glycidyl or oxirane group) as used herein refers to

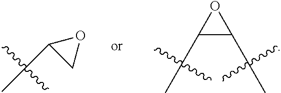

As used herein, the term “flow cell” is intended to mean a vessel having an active area where a reaction can be carried out, a flow channel in fluid communication with the active area, and an inlet for delivering reagent(s) to the flow channel, and an outlet for removing reagent(s) from the flow channel. In some examples, the flow cell accommodates the detection of the reaction that occurs in the flow cell. For example, the flow cell can include one or more transparent surfaces allowing for the optical detection of arrays, optically labeled molecules, or the like.

As used herein, a “flow channel” or “channel” may be an area defined between two bonded components, which can selectively receive a liquid sample. In some examples, the flow channel may be defined between two patterned substrates, and thus may be in fluid communication with the active area(s) of each of the patterned substrates. In other examples, the flow channel may be defined between a patterned substrate and a lid, and thus may be in fluid communication with active area(s) of the patterned substrate.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

As used herein, “heterocycle” means a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The term “hydrazine” or “hydrazinyl” as used herein refers to a —NHNH₂ group.

As used herein, the term “hydrazone” or “hydrazonyl” as used herein refers to a

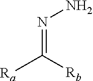

group in which Rₐ and R_{b} are each independently selected from hydrogen, C1-6 alkyl, C2-6 alkenyl, C2-6 alkynyl, C3-7 carbocyclyl, C6-10 aryl, 5-10 membered heteroaryl, and 5-10 membered heterocycle, as defined herein.

As used herein, “hydroxy” or “hydroxyl” refers to an —OH group.

As used herein, the term “interstitial region” refers to an area, e.g., of a patterned substrate that separates depressions and/or separates lanes and/or separates flow channels. For example, an interstitial region can separate one depression of an array from another depression of the array. The depressions that are separated from each other can be discrete, i.e., lacking physical contact with each other. In many examples, the interstitial region is continuous, whereas the depressions are discrete, for example, as is the case for a plurality of depressions defined in or on an otherwise continuous surface. In other examples, the interstitial regions and the depressions are discrete, for example, as is the case for a plurality of lanes in the shape of trenches, which are separated by respective interstitial regions. The separation provided by an interstitial region can be partial or full separation. Interstitial regions may have a surface material that differs from the surface material of the depressions or lanes. For example, depressions or lanes can have the polymer and primers therein, and the interstitial regions can be free of both the polymer and primers.

As used herein, a “nucleotide” includes a nitrogen containing heterocyclic base, a sugar, and one or more phosphate groups. Nucleotides are monomeric units of a nucleic acid sequence. In RNA (ribonucleic acid), the sugar is a ribose, and in DNA (deoxyribonucleic acid), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

In some examples, the term “over” may mean that one component or material is positioned directly on another component or material. When one is directly on another, the two are in contact with each other. In other examples, the term “over” may mean that one component or material is positioned indirectly on another component or material. By indirectly on, it is meant that a gap or an additional component or material may be positioned between the two components or materials.

A “patterned substrate” refers to a support material that includes the active area material(s) in a pattern, e.g., in depressions or in lanes. In some examples, the substrate is exposed to patterning techniques (e.g., etching, lithography, etc.) in order to generate the pattern for the active areas.

The term “polymeric hydrogel” refers to a semi-rigid polymer that is permeable to liquids and gases. The polymeric hydrogel can swell when liquid (e.g., water) is taken up and that can contract when liquid is removed, e.g., by drying. While a hydrogel may absorb water, it is not water-soluble.

As used herein, the “primer” is defined as a single stranded nucleic acid sequence (e.g., single stranded DNA). Some primers, referred to herein as amplification primers, serve as a starting point for template amplification and cluster generation. Other primers, referred to herein as sequencing primers, serve as a starting point for DNA synthesis. The 5′ terminus of the primer may be modified to allow a coupling reaction with a functional group of a polymer. The primer length can be any number of bases long and can include a variety of non-natural nucleotides. In an example, the sequencing primer is a short strand, ranging from 10 to 60 bases, or from 20 to 40 bases.

The term “primer set” refers to a pair of primers that together enable the amplification of a single stranded template nucleic acid strand. Opposed ends of the template strand include adapters to hybridize to the respective primers in a set.

As used herein, the term “protective coating” refers to a water-soluble material in the form of a solid (e.g., a thin film) or a gel that is applied on the active area of a substrate. The protective coating may be any water-soluble material that does not deleteriously affect the underlying surface chemistry (e.g., polymer, primers) or substrate and that serves to protect and/or preserve the functionality of the active area. A water-soluble protective coating is, by definition, distinguishable from the polymer in the active area, as the protective coating dissolves when exposed to water, and may be washed away in this manner; while the polymer is water-insoluble. When the polymer is a hydrogel, the protective coating may swell the hydrogel and at least substantially prevent the layer from undergoing deleterious changes during processing and/or shipping and/or storage. For another example, the protective coating may preserve the accessibility of the primer and at least substantially prevent degradation of the polymer.

A “spacer layer,” as used herein, refers to a material that bonds two components together. In some examples, the spacer layer can be a radiation-absorbing material that aids in bonding, or can be put into contact with a radiation-absorbing material that aids in bonding.

A “subset” of depressions refers to at least two of the depressions of a patterned substrate but less than all of the depressions of the patterned substrate.

The term “substrate” refers to the single layer base support or a multi-layer structure upon which the active area is introduced.

A “thiol” functional group refers to —SH.

As used herein, the terms “tetrazine” and “tetrazinyl” refer to six-membered heteroaryl group comprising four nitrogen atoms. Tetrazine can be optionally substituted.

The term “transparent” when describing a material (e.g., substrate, layer, etc.) means that that the material allows light of a particular wavelength or range of wavelengths to pass through. For example, the material may be transparent to wavelength(s) that are used to chemically change a negative photoresist. Transparency may be quantified using transmittance, i.e., the ratio of light energy falling on a body to that transmitted through the body. The transmittance of a transparent material will depend upon the thickness of the material and the wavelength of light. In the examples disclosed herein, the transmittance of the transparent material may range from 0.25 (25%) to 1 (100%). The material may be a pure material, a material with some impurities, or a mixture of materials, as long as the resulting material is capable of the desired transmittance. Additionally, depending upon the transmittance of the material, the time for light exposure and/or the output power of the light source may be increased or decreased to deliver a suitable dose of light energy through the transparent material to achieve the desired effect (e.g., generating an insoluble photoresist).

### Patterned Substrates

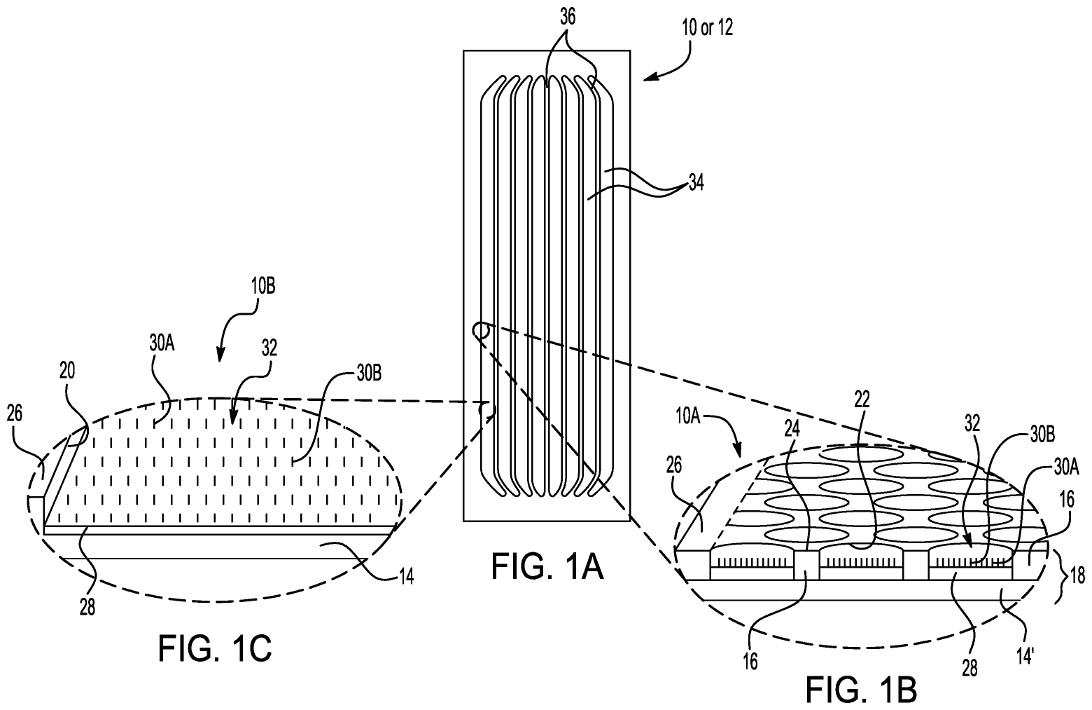

The patterned substrate disclosed herein is suitable for use in a sequencing operation, where single stranded DNA template strands are amplified and sequenced. The patterned substrate may be used in an open format, or may be bonded to a second patterned substrate or to a lid to form a flow cell. A top view of one example of the patterned substrate 10, or of the flow cell 12 including the patterned substrate 10, is depicted in FIG. 1A. Examples of the flow cell 12 will be described in more detail herein below in the section entitled “Flow Cells.”

The patterned substrate 10 includes a single layer base support 14 (as shown in FIG. 1C) or a multi-layered structure 18 (as shown in FIG. 1B). A lane 20 (as shown in FIG. 1C) or a plurality of depressions 22 (as shown in FIG. 1B) may be defined in the single layer base support 14 or an outermost layer (e.g., layer 16) of the multi-layered structure 18.

Examples of suitable single layer base supports 14 include epoxy siloxane, glass, modified or functionalized glass, polymers (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, polyamides, etc.), ceramics/ceramic oxides, silica, fused silica, silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, or the like.

In some examples, as described further in reference to FIG. 5, the patterned substrate 10 is incorporation into a flow cell with a complementary metal-oxide semiconductor (CMOS) chip. In these examples, the single layer base support 14 may be selected so that provides one level of corrosion protection for an embedded metal layer of the CMOS chip that is closest in proximity to the patterned substrate 10. In this example, the single layer base support 14 may include a passivation material that is transparent to the light emissions (e.g., visible light) resulting from reactions at active areas 32, and that is at least initially resistant to the fluidic environment and moisture that may be introduced into or present in the flow channel. An at least initially resistant material acts as an etch barrier to high pH reagents (e.g., pH ranging from 8 to 14) and as a moisture barrier. Examples of suitable passivating materials for the single layer base support 14 include silicon nitride (Si₃N₄), silicon oxide (SiO₂), tantalum pentoxide (TaO₅), hafnium oxide (HaO₂), boron doped p+ silicon, or the like.

Examples of the multi-layered structure 18 include a base support 14′ and at least one other layer 16 thereon, as shown in FIG. 1B.

Some examples of the multi-layered structure 18 include glass or silicon as the base support 14′, with a patterned inorganic oxide as the layer 16. Examples of suitable inorganic oxides include tantalum oxide (e.g., tantalum pentoxide or another tantalum oxide(s) (TaOₓ)), aluminum oxide (e.g., Al₂O₃), silicon oxide (e.g., SiO₂), or hafnium oxide (e.g., HfO₂).

Other examples of the multi-layered structure 18 include the base support 14′ (e.g., glass, silicon, tantalum pentoxide, or any of the other base support 14 materials) and a patterned resin as the other layer 16. It is to be understood that any material that can be selectively deposited, or deposited and patterned to form depressions 22 separated by interstitial regions 24 may be used for the patterned resin. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof. As used herein, the term “polyhedral oligomeric silsesquioxane” (an example of which is commercially available under the tradename “POSS” from Hybrid Plastics) refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for POSS include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

The base support 14, 14′ (whether used singly or as part of the multi-layered structure 18) may be a circular sheet, a panel, a wafer, a die etc. having a diameter ranging from about 2 mm to about 300 mm, e.g., from about 200 mm to about 300 mm, or may be a rectangular sheet, panel, wafer, die etc. having its largest dimension up to about 10 feet (˜ 3 meters). The larger forms of the base support 14, 14′ may be flexible rolls that enable roll to roll processing. For example, a die may have a width ranging from about 0.1 mm to about 10 mm. While example dimensions have been provided, it is to be understood that a single base support 14 with any suitable dimensions may be used.

The example of the patterned substrate 10A shown in FIG. 1B includes depressions 22 defined in the layer 16 of the multi-layered structure 18. Alternatively, the depressions 22 may be defined in the single layer base support 14. Each depression 22 defines a discrete area of the patterned substrate 10A where an active area 32 can be formed.

The depressions 22 may be defined via etching, imprinting, lithography, or another suitable technique.

As one example, depressions 22 may be etched into a glass single layer base support 14.

As another example, the inorganic oxide may be selectively applied to the base support 14′ via vapor deposition, aerosol printing, or inkjet printing to generate the layer 16 having the depressions 22 defined therein.

As still another example, the polymeric resin may be applied to the base support 14′ and then patterned to generate the layer 16 having the depressions 22 defined therein. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, etc.

Many different layouts of the depressions 22 may be envisaged, including regular, repeating, and non-regular patterns. In an example, the depressions 22 are disposed in a hexagonal grid for close packing and improved density. Other layouts may include, for example, rectilinear (rectangular) layouts, triangular layouts, and so forth. In some examples, the layout or pattern can be an x-y format in rows and columns. In other examples, the layout or pattern can be a repeating arrangement of depressions 22 and the interstitial regions 24. In still other examples, the layout or pattern can be a random arrangement of the depressions 22 and the interstitial regions 24.

The layout or pattern may be characterized with respect to the density (number) of the depressions 22 in a defined area. For example, the depressions 22 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the depressions 22 separated by less than about 100 nm, a medium density array may be characterized as having the depressions 22 separated by about 400 nm to about 1 μm, and a low density array may be characterized as having the depressions 22 separated by greater than about 1 μm.

The layout or pattern of the depressions 22 may also or alternatively be characterized in terms of the average pitch, or the spacing from the center of one depression 22 to the center of an adjacent depression 22 (center-to-center spacing) or from the right edge of one depression 22 to the left edge of an adjacent depression 22 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of can be between one of the lower values and one of the upper values selected from the ranges above. In an example, the depressions 22 have a pitch (center-to-center spacing) of about 1.5 μm. While example average pitch values have been provided, it is to be understood that other average pitch values may be used.

The size of each depression 22 may be characterized by its volume, opening area, depth, and/or diameter or length and width. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10−3 μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and width can range from about 0.1 μm to about 100 μm, e.g., about 0.5 μm, about 1 μm, about 10 μm, or more, or less.

A portion (e.g., the perimeter) of the single base support 14 or of the layer 16 in which the depressions 22 are formed may remain unpatterned and available for attachment to a lid (not shown) or another patterned substrate. These portions are referred to as bonding regions 26 (e.g., as shown in FIG. 1B). It is to be further understood that the patterned substrate 10A may include several subsets of depressions 22 defined in the single base support 14 or in the layer 16, and each subset may be surrounded by an unpatterned region of the base support 14 or layer 16. As an example, in FIG. 1A, the areas 34 can represent regions of the patterned substrate 10A that include respective subsets of depressions 22, and the areas 36 can represent unpatterned regions that physically separate the subsets of depression 22. These unpatterned regions can be used as bonding regions 26.

The example of the patterned substrate 10B shown in FIG. 1C includes a lane 20 defined in the single layer base support 14. In other examples, the lane 20 may be defined in the layer 16 of the multi-layered structure 18. The lane 20 defines one continuous active area 32.

The depth of the lane 20 is less than the thickness of the component (e.g., base support 14 or layer 16) in which the lane 20 is defined.

The lane 20 may have any suitable geometric configuration, such as a rectangular configuration or a substantially rectangular configuration with rounded ends. The length and width of the lane 20 may be selected so that an unpatterned portion of the single base support 14 or of the layer 16 surrounds the lane 20. This surrounding portion may be a bonding region 26 (see FIG. 1C), which is available for attachment to a lid (not shown) or another patterned substrate. It is to be further understood that the patterned substrate 10B may include several lanes 20 defined in the single base support 14 or in the layer 16, and each lane 20 may be surrounded by an unpatterned region of the base support 14 or layer 16. As an example, in FIG. 1A, the areas 34 can represent regions of the patterned substrate 10B that include a single lane 20, and the areas 36 can represent unpatterned regions that physically separate the lanes 20. These unpatterned regions (e.g., areas 36) can be used as bonding regions 26.

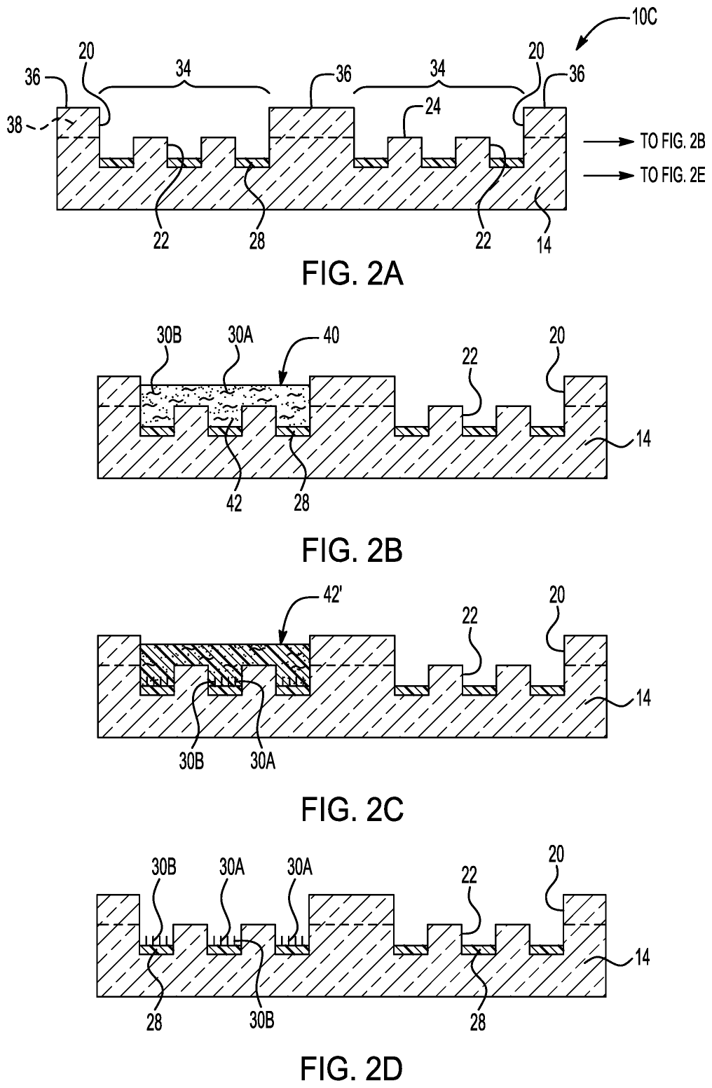

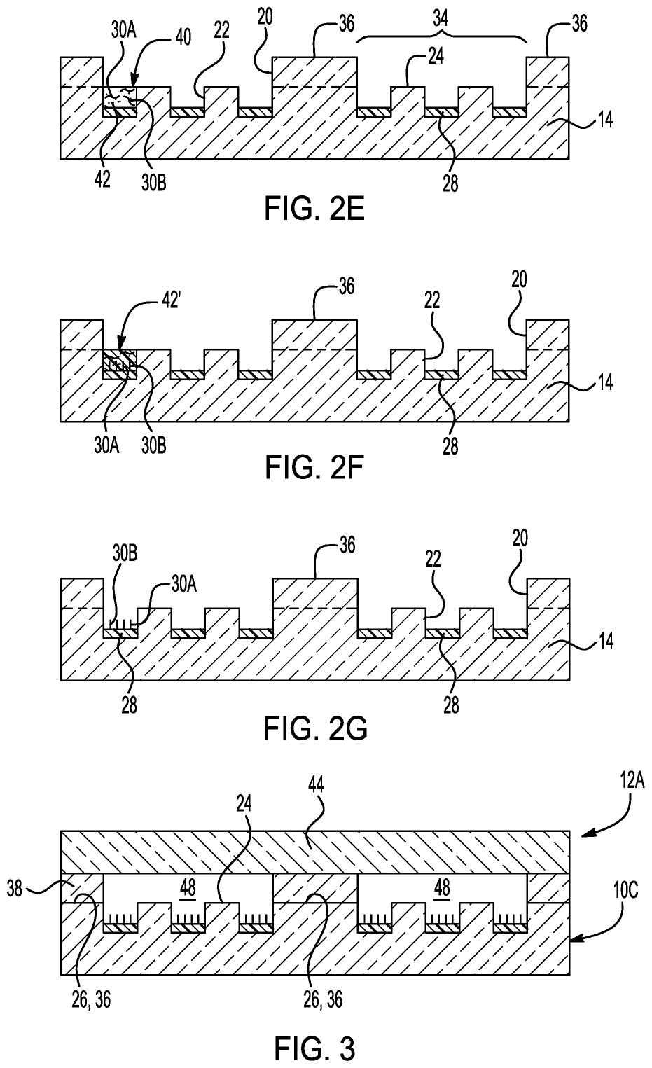

In other examples, the patterned substrate 10 includes both the lane 20 and the depressions 22, which are patterned within the lane 20. This patterned substrate 10C is shown in FIG. 2A. In some examples, a portion of the base support 14 or layer 16 is etched or imprinted or otherwise patterned to form the lane 20, and then the depressions 22 are patterned in the portion of the base support 14 or layer 16 that is positioned between the walls of the lane 20. In other examples, the depressions 22 are patterned in the base support 14 or layer 16 (e.g., as shown in FIG. 1B), and then an additional material (e.g., material 38 in FIG. 3 through FIG. 5) may be selectively deposited on a portion to form the walls that partially define the lane 20. When a flow cell 12 is to be formed, the material 38 may be an adhesive or a radiation-absorbing material that aids in bonding.

Each of the architectures shown in FIG. 1B and FIG. 1C also includes the active area 32. The active area 32 includes the polymer 28 and primers 30A, 30B. In the patterned substrate 10A of FIG. 1B, respective active areas 32 are located within the depressions 22. In the patterned substrate 10B of FIG. 1C, the active area 32 extends along the lane 20.

The polymer 28 may be any polymeric material that includes a first click reaction moiety (e.g., R^{A} in structure (I) below) that is capable of undergoing copper-free click chemistry with a second click reaction moiety that is attached to the primers 30A, 30B. In one example, the polymer 28 is a hydrogel, which can swell when liquid is taken up and can contract when liquid is removed, e.g., by drying. Other suitable polymers 28 include self-assembled monolayers.

In an example, the polymer 28 is an acrylamide copolymer, such as poly(N-(5-azidoacetamidylpentyl)acrylamide-co-acrylamide, PAZAM. PAZAM and some other forms of the acrylamide copolymer are represented by the following structure (I):

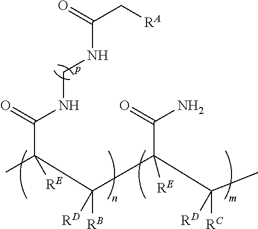

wherein:
R^{A} is selected from the group consisting of azide and tetrazine;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.
   These acrylamide copolymers are hydrogels.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are lightly cross-linked polymers.

In other examples, the gel material may be a variation of the structure (I). In one example, the acrylamide unit may be replaced with N,N-dimethylacrylamide

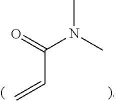

In this example, the acrylamide unit in structure (I) may be replaced with

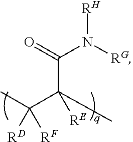

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

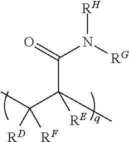

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymer 28, the recurring “n” feature in structure (I) may be replaced with a monomer including a heterocyclic azido group having structure (II):

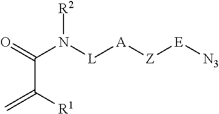

wherein R¹ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the polymer 28 may include a recurring unit of each of structure (III) and (IV):

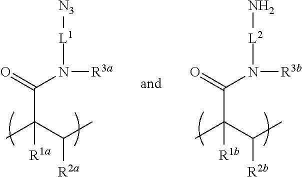

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

It is to be understood that other polymers 28 may be used, as long as they are functionalized, e.g., with the azide or tetrazine, to graft oligonucleotide primers 30A, 30B thereto. Some examples of other polymers 28 include polysilanes having functional groups (i.e., azide or tetrazine) that can attach the desired primer set 30A, 30B, or an azidolyzed version of silane free acrylamide (SFA). A variety of polymer architectures containing acrylamide may be utilized in the examples disclosed herein, such as branched polymers, including dendrimers, and the like. For example, the monomers (e.g., acrylamide, etc.) may be incorporated, either randomly or in block, into the branches (arms) of a dendrimer.

The polymer 28 may be formed using any suitable copolymerization process. The polymer 28 may also be applied using any suitable deposition technique. As examples, depositing may be performed using vapor deposition techniques, coating techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, microcontact printing, or the like. These deposition techniques apply the polymer 28 over the surface of the base support 14 or the outermost layer 16 of the multi-layer structure 18, including in the depressions 22 or in the lane 20.

The attachment of the polymer 28 to the underlying base support 14 or layer 16 (e.g., a metal oxide coating, a resin, etc.) of the multi-layer structure 18 may be through covalent bonding. In some instances, the underlying base support 14 or layer 16 may first be activated, e.g., through silanization or plasma ashing. Covalent linking of the polymer 28 is helpful for maintaining the primers 30A, 30B in the active area 32 throughout the lifetime of the patterned substrate 10 and/or flow cell 12 during a variety of uses.

The polymer 28 that is positioned over the interstitial regions 24 and/or the bonding region 26 may be removed, e.g., using a polishing process. The polishing process may be performed with a chemical slurry, which can remove the polymer 28 from the interstitial regions 24 and the bonding region 26 without deleteriously affecting the underlying substrate at those regions 24, 26. An example of the chemical slurry may include abrasive particles, a buffer, a chelating agent, a surfactant, and/or a dispersant. Alternatively, polishing may be performed with a solution that does not include the abrasive particles.

The chemical slurry may be used in a chemical mechanical polishing system to polish the surface of the interstitial regions 24 and the bonding region 26. The polishing head(s)/pad(s) or other polishing tool(s) is/are capable of polishing the polymeric hydrogel 28 that may be present over the interstitial regions 24 and the bonding region 26 while leaving the polymer 28 in the depression(s) 22 at least substantially intact. As an example, the polishing head may be a Strasbaugh ViPRR II polishing head.

Each of the architectures also includes the primers 30A, 30B attached to the polymer 28. The primers 30A, 30B are part of a set that enables the amplification of a template nucleic acid strand. As examples, the primers 30A, 30B may include P5 and P7 primers, P15 and P7 primers, or any combination of the PA primers, the PB primers, the PC primers, and the PD primers set forth herein. As further examples, any two PA, PB, PC, and PD primers may be used together, or any combination of one PA primer and one PB, PC, or PD primer may be used, or any combination of one PB primer and one PC or PD primer may be used, or any combination of one PC primer and one PD primer may be used.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, NOVASEQX™, GENOME ANALYZER™, ISEQ™, and other instrument platforms. The P5 primer may be:

| | P5 #1: 5′ → 3′ |
|---|---|
| (SEQ. ID. NO. 1) | |
| | AATGATACGGCGACCACCGAGAUCTACAC; |
| | P5 #2: 5′ → 3′ |
| (SEQ. ID. NO. 2) | |
| | AATGATACGGCGACCACCGAGAnCTACAC |
| | where “n” is inosine in SEQ. |
| | ID. NO. 2; |
| | or |
| | P5 #3: 5′ → 3′ |
| (SEQ. ID. NO. 3) | |
| | AATGATACGGCGACCACCGAGAnCTACAC |
| | where “n” is alkene-thymidine |
| | (i.e., alkene-dT) in SEQ. ID. |
| | NO. 3. |

The P7 primer may be any of the following:

| | P7 #1: 5′ → 3′ |
|---|---|
| (SEQ. ID. NO. 4) | |
| | CAAGCAGAAGACGGCATACGAnAT; |
| | P7 #2: 5′ → 3′ |
| (SEQ. ID. NO. 5) | |
| | CAAGCAGAAGACGGCATACnAGAT; |
| | or |
| | P7 #3: 5′ → 3′ |
| (SEQ. ID. NO. 6) | |
| | CAAGCAGAAGACGGCATACnAnAT |
| | where “n” is 8-oxoguanine |
| | in each of the sequences |
| | 4 through 6. |

The P15 primer is:

| | P15: 5′ → 3′ |
|---|---|
| (SEQ. ID. NO. 7) | |
| | AATGATACGGCGACCACCGAGAnCTACAC |
| | where “n” is allyl-T (i.e., a |
| | thymine nucleotide analog |
| | having an allyl functionality). |

The other primers (PA-PD) mentioned above include:

| | PA 5′ → 3′ |
|---|---|
| (SEQ. ID. NO. 8) | |
| | GCTGGCACGTCCGAACGCTTCGTTAATCCGTTGAG |
| | PB 5′ → 3′ |
| (SEQ. ID. NO. 9) | |
| | CGTCGTCTGCCATGGCGCTTCGGTGGATATGAACT |
| | PC 5′ → 3′ |
| (SEQ. ID. NO. 10) | |
| | ACGGCCGCTAATATCAACGCGTCGAATCCGCAACT |
| | PD 5′ → 3′ |
| (SEQ. ID. NO. 11) | |
| | GCCGCGTTACGTTAGCCGGACTATTCGATGCAGC |

While not shown in the example sequences for PA-PD, it is to be understood that any of these primers may include a cleavage site, such as uracil, 8-oxoguanine, allyl-T, etc. at any point in the strand. When used together in a primer set, the cleavage sites of the PA-PD primers are orthogonal so that they are susceptible to different cleaving agents. Thus, the cleavage of amplicons from one of the primers in the set (e.g., PA) does not result in cleavage of amplicons from the other of the primers in the set (e.g., PB, PC, or PD). In any of the primer sets disclosed herein, the primers have orthogonal cleavage sites.

Each of the primers 30A, 30B disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ end of each primer 30A, 30B includes the second click reaction moiety that is capable of undergoing copper-free click chemistry with the first click reaction moiety that is attached to the polymer 28. The second click reaction moiety is an alkyne-containing linker or an alkene-containing linker. The alkyne in the alkyne-containing linker is to react with an azide click reaction moiety of the polymer 28, or the alkene in the alkene-containing linker is to react with a tetrazine click reaction moiety of the polymer 28. The alkyne is a strained alkyne, where the alkyne is part of a cyclic compound. Bicyclo[6.1.0]nonyne (BCN) and dibenzocyclooctyne (DBCO) are examples that include an internal alkyne. An example of an alkene-containing linker is trans-cyclooctene. In some examples, the first click reaction moiety is azide and the second click reaction moiety is bicyclononyne, or the first click reaction moiety is azide and the second click reaction moiety is dibenzocyclooctyne, or the first click reaction moiety is tetrazine and the second click reaction moiety is trans-cyclooctene.

The primers 30A, 30B are immobilized on the polymer 28 as described hereinbelow.

While primers 30A, 30B are described herein, it is to be understood that other biomolecules may be used in place of the primers 30A, 30B. The biomolecule selected will depend, in part, upon the intended use of the patterned substrate 10. In any example, the biomolecule includes the second click reaction moiety for undergoing a copper free click reaction with the first click reaction moiety of the polymer 28. Some specific examples of other biomolecules include biotin, proteins (e.g., streptavidin), enzymes (e.g., polymerase), DNA, RNA, aptamers, oligonucleotides used for quality control but are non-functional in amplification and sequencing, or some macromolecular combination of these. It is to be understood that the quality control oligonucleotides may be used with the primers 30A, 30B.

### Biomolecule Immobilization

After the lane 20 or the depressions 22 are generated in the single layer base support 14′ or the layer 16 of the multi-layered substrate 18 and the polymer 28 is applied to the lane 20 or the depressions 22, the primers 30A, 30B are immobilized to the polymer 28 using the method disclosed herein. The method generally includes: i) using a liquid-phase thin-film deposition technique, applying a grafting solution to the patterned substrate 10 (e.g., 10A, 10B), wherein: a) the patterned substrate 10 includes a lane 20 surrounded by interstitial regions 24 (which may be the bonding region 26) or a plurality of depressions 22 separated by interstitial regions 24; and the polymer 28 in the lane 20 or in each of the plurality of depressions 22, the polymer 28 being functionalized with the first click reaction moiety; and b) the grafting solution includes a solvent, a polymer matrix material dissolved in the solvent; and primers 30A, 30B of the primer set dissolved in the solvent, each of the primers 30A, 30B being terminated with a second click reaction moiety; and ii) drying the grafting solution, thereby forming a solid polymer matrix 42′ (see FIG. 2C and FIG. 2F) and attaching at least some of the primers 30A, 30B to the polymer 28 i) via the first and second click reaction moieties and ii) in at least a portion of the lane 20 or in at least some of the plurality of depressions 22.

Examples of this method are shown and described in reference to FIG. 2A through FIG. 2D and FIG. 2A and FIG. 2E through FIG. 2G.

FIG. 2A depicts an example of the patterned substrate 10C including the single layer base support 14 having multiple lanes 20 defined therein, and having multiple depressions 22 defined within each lane 20. While the single layer base support 14 is depicted, it is to be understood that the multi-layered substrate 18 may be used instead. As described herein, the walls of the lanes 20 may be entirely defined by the single layer base support 14 (or the layer 16 if the multi-layered substrate 18 is used), or by a separate material 38 that is applied to the areas 36 of the patterned substrate 10 that separate the lanes 20 (and in this example, each subset of depressions 22).

Each of the example methods shown in FIG. 2A through FIG. 2D and in FIG. 2A and FIG. 2E through FIG. 2G utilizes the grafting solution. The grafting solution is shown at reference numeral 40 in FIG. 2B and in FIG. 2F. The grafting solution 40 includes the solvent, the polymer matrix material 42 dissolved in the solvent, and the primers 30A, 30B dissolved in the solvent.

The solvent may be any liquid that can dissolve both the polymer matrix material 42 and the primers 30A, 30B. An example of a suitable solvent is water. Other examples of suitable solvents include alcohols (e.g., methanol, ethanol, propanol, etc.), glycols (e.g., ethylene glycol, propylene glycol, etc.), glycol ethers (e.g., propylene glycol propyl ether, propylene glycol methyl ether, propylene glycol phenyl ether, and propylene glycol dipropyl ether, and oligomeric forms of ethylene glycol (e.g., diethylene glycol, triethylene glycol, etc.). Depending upon the solubility characteristics of the polymer matrix material 42 and the primers 30A, 30B, the solvent may include a combination of water and the alcohol(s), or the glycol(s), or the oligomeric form of ethylene glycol, where the alcohol(s), or the glycol(s), or the oligomeric form of ethylene glycol is present in an amount up to 60% (v/v). The alcohol(s), or the glycol(s), or the oligomeric form of ethylene glycol may help to increase the drying rate, decrease the surface tension, and/or improve thin film uniformity. Other suitable co-solvents may include low volatility solvents, such as glycerol or dimethyl sulfoxide, to slow down evaporation, which may be desirable, for example, to allow more time for self-assembly or phase separation. In an example, it may be desirable to select a hygroscopic co-solvent, such as glycerol, butanol, and/or lithium chloride. Poly(ethylene glycol) may also be used.

The polymer matrix material is selected from the group consisting of a water-soluble non-cationic synthetic polymer; a water-soluble natural polysaccharide or a derivative thereof; a water-soluble natural protein or a derivative thereof; and combinations thereof.

Examples of the water-soluble non-cationic synthetic polymer include polyacrylamides, poly(acrylic acid), poly(methacrylic acid), poly(vinyl pyrrolidone), poly(vinyl alcohol), poly(methacrylamide), poly(N-alkyl acrylamide)s, poly(N-dialkyl acrylamide)s, poly(N-(2-hydroxypropyl) methacrylamide), poly(divinyl ether-maleic anhydride), poly(phosphates), poly(2-alkyl-2-oxazolines), poly(hydroxyethyl methacrylate), poly(sulfobetaine methacrylate), silicones, polyacrylates (e.g., sodium polyacrylate), polyethers (e.g., polyvinyl ethers, polyethylene glycol, etc.), poly(vinyl ether-maleic acid), poly(2-hydroxyethyl acrylate), hydroxyl functional polymers, polypeptides (e.g., poly(glutamic acid) and its salts), or derivatives of the aforementioned polymers including, for example, random, block and graft copolymers and branched analogues. An example of a suitable copolymer is a polyvinyl alcohol/polyethylene glycol graft copolymer (one example of which includes KOLLICOAT® IR, available from BASF Corp.). An example of a suitable hydroxyl functional polymer is commercially available from BASF Corp. under the tradename KOLLICOAT® PR. Any of the water-soluble non-cationic synthetic polymers that include acid groups may be used in their alkali metal salt form.

Some specific examples of the natural polysaccharides or their derivatives thereof include starch, carboxymethylcellulose, xanthan gum, pectin, dextran, carrageenan, guar gum, cellulose, pullulan, hydroxypropylmethyl cellulose (HPMC) hydroxypropyl cellulose (HPC), hydroxyethyl cellulose (HEC), methyl cellulose, carboxymethylhydroxyethyl cellulose (CMHEC), hyaluronic acid, starch phosphate, hydroxypropyl starch, hydroxyethyl starch, agarose, agar, and alginate.

Some specific examples of the natural proteins thereof include casein and albumin. Derivatives of these natural proteins may also be used.

The primers 30A, 30B may be any of the examples set forth herein.

In an example, the grafting solution 40 includes up to 15% (mass to volume) of the polymer matrix material 42; and up to 10% (mass to volume) of the primers 30A, 30B. In one example, the grafting solution 40 includes from about 5% (mass to volume) up to 15% (mass to volume) of the polymer matrix material 42. In another example, the grafting solution 40 includes from about 12% (mass to volume) to about 13% (mass to volume) of the polymer matrix material 42. In one example, the grafting solution 40 includes up to about 1% (mass to volume) of the primers 30A, 30B. In another example, the grafting solution 40 includes from about 0.25% (mass to volume) to about 0.75% (mass to volume) of the primers 30A, 30B.

Some examples of the grafting solution 40 further include an additive. In one example, the grafting solution 40 further includes an additive selected from the group consisting of a sugar, a sugar alcohol, a surfactant, and combinations thereof. An example of a suitable sugar includes sucrose. An example of a suitable sugar alcohol includes sorbitol. Examples of suitable surfactants include water soluble anionic or non-ionic surfactants. An example of a suitable anionic surfactant includes sodium dodecyl sulfate. Examples of suitable non-ionic surfactants include polysorbates, such as polysorbate 20 (e.g., TWEEN™ 20), or sorbitan laurate (e.g., SPAN™ 20), or oxyethylene oligomers with an n-alkane tail (e.g., the BRIJ® family of non-ionic surfactants, such as BRIJ®-35).

The grafting solution 40 may include up to 35% (mass to volume) of the additive(s), whether used alone or in combination. Thus, if a single additive (i.e., the sugar or the surfactant) is included, the total is up to 35% (mass to volume). Similarly, if both the sugar and the surfactant are included, the total is up to 35% (mass to volume). Additionally, when the additive(s) is/are included, the total (mass to volume) of the polymer matrix material 42 and the additive(s) ranges from about 5% to about 40%. Thus, examples of the grafting solution include up to 15% (mass to volume) of the polymer matrix material; up to 35% (mass to volume) of the additive(s); and up to 10% (mass to volume) of the primers; where a total (mass to volume) of the polymer matrix material 42 and the additive(s) ranges from about 5% to about 40%.

In one example of the grafting solution 40, the polymer matrix material 42 includes the water-soluble non-cationic synthetic polymer, the water-soluble non-cationic synthetic polymer is the polyvinyl alcohol/polyethylene glycol graft copolymer, the additive is the sugar, and the sugar is sucrose. In this example, the polyvinyl alcohol/polyethylene glycol graft copolymer may range from 5% to about 15% (mass to volume) and the sucrose may range from about 1% to about 25% (mass to volume), where a total (mass to volume) of the polymer matrix material 42 and the sucrose and the ranges from about 5% to about 30%.

In examples, the grafting solution 40 consists of water, the optional co-solvent, the polymer matrix material 42, the listed additive(s), and the primers 30A, 30B. Thus, in these examples, additional components (e.g., salt(s)) are not included.

FIG. 2B and FIG. 2E depict two examples of the application of the grafting solution 42. A liquid-phase thin-film deposition technique is used that enables the selective deposition of the grafting solution 42 with high precision. Examples of suitable liquid-phase thin-film deposition techniques that may be used include dispense coating, slot-die coating, inkjet printing, spray coating, screen printing, blade coating, roll coating, gravure coating, and dip coating. Dispense coating may be performed with a precision gantry tool.

As one example, the grafting solution 42 may be dispensed in a line having a width ranging from about 300 μm to about 1 mm. The precision of the dispensed line renders this method particularly desirable for dispensing the grafting solution 40 in each lane 20 of the patterned substrates 10B, 10C, as shown in FIG. 2B, or in at least some of the depressions 22 of the patterned substrate 10A. In one example, each lane 20 or all of the depressions 22 may have the same grafting solution 40 applied thereto. In another example, each lane 20 or each subset of depressions 22 may have a different grafting solution 40 applied thereto so that a different primer set (or biomolecule) may be introduced into each of the lanes 20 or depression subsets. This enables the formation of a multi-functional patterned substrate 10A, 10B, 10C. In the example where different grafting solutions 40 are applied to different subsets of depressions 22, the number of depressions 22 in a particular subset and the geometric configuration of that subset may be determined, in part, by the precision capability of the liquid-phase thin-film deposition technique that is used. As examples, precision dispense coating or slot-die coating may be used to form a subset that receives one specific grafting solution 40 where the depressions 22 in the subset are in a line, a rectangular shape, etc.

As another example, the grafting solution 40 may be dispensed as dots having a diameter ranging from about 0.5 mm to about 2 mm. The precision of the dispensed dots renders this method particularly desirable for dispensing the grafting solution 40 in larger depressions 22. In one example, each depression 22 may have the same grafting solution 40 applied thereto. In another example, each depression 22 may have a different grafting solution 40 applied thereto so that a different primer set (or biomolecule) may be introduced into each of the depressions 22.

Using the liquid-phase thin-film deposition techniques disclosed herein, the bonding region(s) 26 remains free of the grafting solution 40, and thus the method may reduce material waste.

The method then involves drying the grafting solution 40. In one example, drying the grafting solution 40 is accomplished at ambient temperature (e.g., 18° C. to 26° C.). In another example, drying the grafting solution 40 involves exposing the applied grafting solution 40 to heat ranging from about 30° C. to about 120° C. In another example, the heat may range from about 40° C. to about 120° C. In still another example, the heat may range from about 60° C. to about 100° C. In one specific example, the heat may be about 80° C. Heat exposure may take place in an oven, on a hot plate, using an infrared heat source, or using any other suitable technique.

The drying rate may also be controlled by controlling the humidity in the drying environment or drying under an inert gas, such as N₂ or Ar.

During drying, the polymer matrix material 42 solidifies to form a solid polymer matrix 42′, as shown in FIG. 2C and FIG. 2F. Depending upon the polymer matrix material 42 that is used in the grafting solution 40, drying may also result in curing (e.g., a change in the morphology and/or physical properties) of the polymer matrix material 42. Additional heating may be used in order to ensure the resulting solid polymer matrix 42′ exhibits a desirable morphology.

Also during drying, the first and second click reaction moieties undergo a copper-free click chemistry reaction. It is believed that in the solid state of the solid polymer matrix 42′, this click reaction takes place more rapidly than in the liquid state. The click reaction results in at least some of the primers 30A, 30B being covalently attached to the polymer 28 in the lane and/or in at least some of the depressions 22. It is to be understood that some of the primers 30A, 30B in the grafting solution 42 may not undergo the click reaction, but may remain dispersed in the solid polymer matrix 42′ that is formed. As will be described in more detail below, these primers 30A, 30B may be removed at a desired time with the solid polymer matrix 42′.

The thickness of the solid polymer matrix 42′ is about 10 μm or less. In one example, the thickness of the solid polymer matrix 42′ ranges from about 5 μm to about 8 μm.

The method shown and described in reference to FIG. 2A through FIG. 2C or FIG. 2A, FIG. 2E, and FIG. 2F may be repeated to introduce the same primer set or a different primer set to uncoated lane(s) 20 and/or depression(s) 22.

In another example method, the grafting solution 42 is introduced into a formed flow cell (see reference numerals 12A, 12B, 12C in FIG. 3 through FIG. 5). In these examples, the grafting solution 40 is flowed into the bonded flow cell 12A, 12B, 12C using a flow through technique and then an air or nitrogen gas bubble is introduced into the flow channel 48 to dry down the grafting solution 40 to form a thin film on the exposed surfaces of the patterned structure(s) 10A, 10B, 10C. Thus, in this example method, the patterned substrate 10A, 10B, 10C is part of a flow cell 12A, 12B, 12C including the patterned substrate 10A, 10B, 10C bonded to a lid 44 or a second patterned substrate 10A, 10B, 10 and a flow channel 48 defined between the patterned substrate 10A, 10B, 10C and the lid 44 or the second patterned substrate 10A, 10B, 10C; the liquid-phase thin-film deposition technique is a flow through technique; and drying the grafting solution 40 involves introducing an air or nitrogen gas bubble into the flow cell 12A, 12B, 12C while the grafting solution 40 is present in the flow channel 48.

### Solid Polymer Matrix Removal

In some examples, the solid polymer matrix 42′ may be allowed to remain in the lane(s) 20 and/or in the depression(s) 22 until it is desirable to utilize the patterned substrate 10A, 10B, 10C for a particular application, such as open substrate sequencing, bonding to form a flow cell 12, sequencing in a flow cell 12, or some other suitable application. In these examples, the solid polymer matrix 42′ serves as a protective coating for the grafted primers 30A, 30B and the polymer 28 until it is removed.

The removal of the solid polymer matrix 42′ may take place i) when it is desirable to perform open substrate sequencing, ii) before the formation of the flow cell 12, or iii) after the formation of a flow cell 12 and before a sequencing operation is performed in the flow cell 12. Thus, examples of the method described herein further include removing the solid polymer matrix 42′ and unattached primers by rinsing the patterned substrate 10A, 10B, 10C with a solvent of the solid polymer matrix 42′, whereby the attached primers 30A, 30B remain in the lane(s) 20 and/or the at least some of the plurality of depressions 22.

Because the solid polymer matrix 42′ is water soluble, water may be used to remove the solid polymer matrix 42′ (or matrices if different grafting solutions 40 are introduced and solidified).

The open (non-bonded) patterned substrates 10C shown in FIG. 2C and FIG. 2F (or any of the open patterned substrates 10A, 10B disclosed herein that have had the solid polymer matrix 42′ formed thereon) may be submerged in water or may have water poured/sprayed thereon to remove the solid polymer matrix 42′. The water wash may be performed for a time ranging from about 3 seconds to about 5 minutes. This will expose the primers 30A, 30B and the polymer 28, rendering the patterned structure 10A, 10B, 10C ready for use in a sequencing operation.

When the solid polymer matrix 42′ is removed after the flow cell 12 is formed, the water may be introduced into the flow cell 12, allowed to incubate, and then removed from the flow cell 12. A flow through method may also be used where water is continuously transported through the flow cell 12 until the solid polymer matrix 42′ is removed. This will expose the primers 30A, 30B and the polymer 28, rendering the flow cell 12 ready for use in a sequencing operation.

In any of these examples, additional water may be used to rinse the patterned substrate 10 (whether it is in its open form or whether it has been bonded to form a flow cell 12). Examples of the patterned substrate 10C after solid polymer matrix 42′ removal are shown in FIG. 2D and FIG. 2G. For simplicity, these examples do not depict the formation of the solid polymer matrix 42′ in the other lane 20 or depressions 22.

### Flow Cells

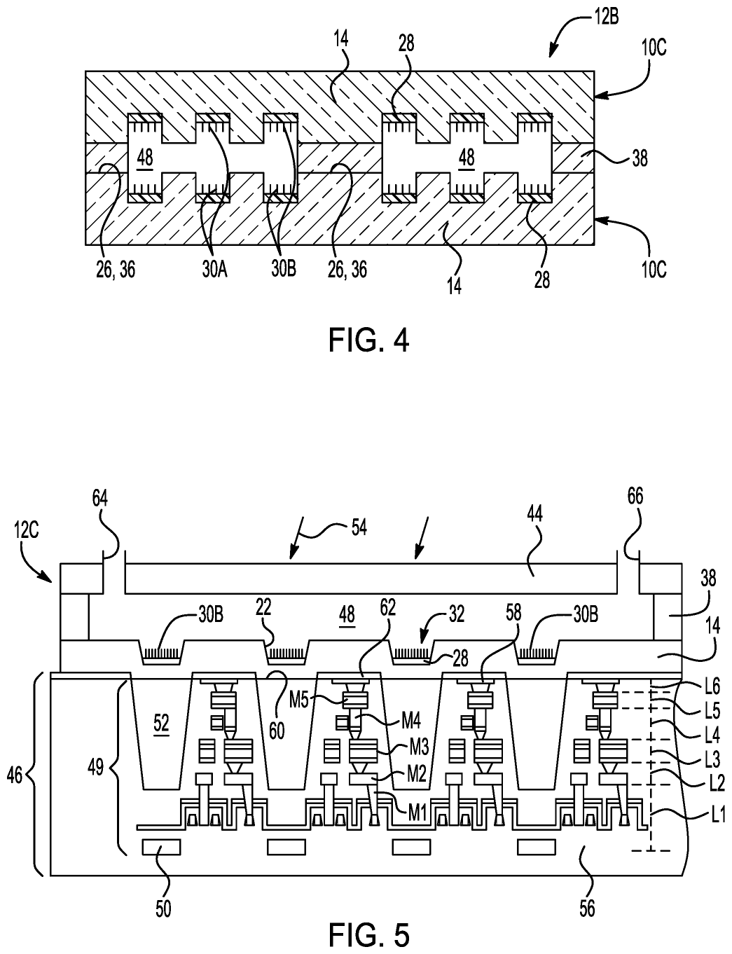

As mentioned, the patterned substrate 10 (e.g., 10A, 10B, 10C) may be incorporated into a flow cell 12, examples of which are shown in FIG. 3, FIG. 4, and FIG. 5. The flow cell 12A depicted in FIG. 3 includes the patterned substrate 10C bonded to a lid 44. The flow cell 12B depicted in FIG. 4 includes two patterned substrates 10C bonded together. The flow cell 12C depicted in FIG. 5 depicts the patterned substrate 10A bonded to a lid 44 and integrated with a CMOS chip 46.

Each example of the flow cell 12A, 12B, 12C includes a flow channel 48 that is formed between the bonded components. The flow cell 12A, 12B, 12C may include multiple flow channels 48 (FIG. 3 and FIG. 4), or may include a single flow channel (FIG. 5). When multiple flow channels 48 are formed, it is to be understood that each flow channel 48 may be isolated from each other flow channel 48 so that fluid introduced into one flow channel 48 does not flow into adjacent flow channel(s) 48. In some examples, the material 38 used for bonding separates adjacent flow channels 48 and forms walls of the flow channels 48.

The depth of the flow channel 48 can be as small as a monolayer thick when microcontact, aerosol, or inkjet printing is used to deposit the material 38 over the bonding region 26 (area 36) that defines the flow channel 48 walls. In other examples, a thicker spacer layer may be applied to bonding region 26 (area 36) so that the spacer layer defines at least a portion of the walls of the flow channel 48. As one example, the spacer layer can be a radiation-absorbing material that aids in bonding. In these examples, the depth of the flow channel 48 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 48 may be greater than, less than or between the values specified above.

The flow cells 12A, 12B, 12C may include inlet and outlet ports (see reference numerals 64 and 66 in FIG. 5) that are configured to fluidically engage other ports (not shown) for directing fluid(s) into the flow channel 48 (e.g., from a reagent cartridge or other fluid storage system component) and out of the flow channel 48 (e.g., to a waste removal system) or a recirculation system.

As mentioned, the flow cell 12A depicted in FIG. 3 includes the patterned substrate 10C bonded to the lid 44. The lid 44 may be any material that is transparent to the excitation light that is to be directed toward the flow cell 12A. In optical detection systems, the lid 44 may also be transparent to the emissions generated from reaction(s) taking place in the flow cell 12A. As examples, the lid 44 may include glass (e.g., borosilicate, fused silica, etc.) or a transparent polymer. A commercially available example of a suitable borosilicate glass is D 263®, available from Schott North America Inc. Commercially available examples of suitable polymer materials, namely cyclo olefin polymers, are the ZEONOR® products available from Zeon Chemicals L.P. In some instances, the lid 44 is shaped to form the top of the flow cell 12A (as shown in FIG. 3), and in other instances, the lid is shaped to form both the top of the flow cell 12A as well as sidewalls of one or more of the flow channels 48.

Any suitable technique, such as laser bonding, diffusion bonding, anodic bonding, eutectic bonding, plasma activation bonding, glass frit bonding, or other methods known in the art may be used to bond the patterned substrate 10A, 10B, 10C and the lid 44 together.

While not shown in FIG. 3, it is to be understood that the solid polymer matrix 42′ generated to graft the primers 30A, 30B to the lane(s) 20 and/or depression(s) 22 may remain in the lane(s) 20 and/or depression(s) 22 while the patterned substrate 10A, 10B, 10C is bonded to the lid 44. Because the application methods disclosed herein can precisely dispense the grafting solution 40, the bonding region(s) 26 remain free of the grafting solution 40 and the resulting solid polymer matrix 42′. It may be desirable for the solid polymer matrix 42′ to remain in the lane(s) 20 and/or depression(s) 22 during bonding to protect the polymer 28 and the attached primers 30A, 30B. In this example, the solid polymer matrix 42′ may be removed (as described herein) from the patterned structure 10A, 10B, 10C of the flow cell 12A before a sequencing operation is performed.

Alternatively, the solid polymer matrix 42′ generated to graft the primers 30A, 30B to the lane(s) 20 and/or depression(s) 22 may be removed from the lane(s) 20 and/or depression(s) 22 before the patterned substrate 10A, 10B, 10C is bonded to the lid 44. In this example, the patterned substrate 10A, 10B, 10C would be exposed to a suitable removal solvent (e.g., water) and rinsed before bonding takes place.

The flow cell 12B depicted in FIG. 4 includes two patterned substrates 10C bonded together. While the structure 10C is depicted, it is to be understood that any example of the patterned structure 10A, 10B, 10C may be bonded together to form a flow cell 12B.

Any suitable technique, such as laser bonding, diffusion bonding, anodic bonding, eutectic bonding, plasma activation bonding, glass frit bonding, or other methods known in the art may be used to bond the patterned substrates 10A, 10B, 10C together.

While not shown in FIG. 4, it is to be understood that the solid polymer matrix 42′ generated to graft the primers 30A, 30B to the lane(s) 20 and/or depression(s) 22 may remain in the lane(s) 20 and/or depression(s) 22 while the patterned substrates 10A, 10B, 10C are bonded together. Because the application methods disclosed herein can precisely dispense the grafting solution 40, the bonding region(s) 26 remain free of the grafting solution 40 and the resulting solid polymer matrix 42′. It may be desirable for the solid polymer matrix 42′ to remain in the lane(s) 20 and/or depression(s) 22 during bonding to protect the polymer 28 and the attached primers 30A, 30B. In this example, the solid polymer matrices 42′ may be removed (as described herein) from the patterned structures 10A, 10B, 10C of the flow cell 12B before a sequencing operation is performed.

Alternatively, the solid polymer matrix 42′ generated to graft the primers 30A, 30B to the lane(s) 20 and/or depression(s) 22 may be removed from the lane(s) 20 and/or depression(s) 22 before the patterned substrates 10A, 10B, 10C are bonded together. In this example, the respective patterned substrate 10A, 10B, 10C would be exposed to a suitable removal solvent (e.g., water) and rinsed before bonding takes place.

The flow cell 12C depicted in FIG. 5 depicts the patterned substrate 10A bonded to a lid 44 and integrated with a CMOS chip 46. This particular flow cell 12C is integrated over a solid-state imager, such as the complementary metal-oxide semiconductor (CMOS) imager, which does not require a large optical assembly to detect the fluorescent emissions from each of the active areas 32.

In this example, any example of the patterned substrates 10A, 10B, 10C may be bonded to the lid 44 as described in reference to FIG. 3. In this example, the base support 14 is one of the passivation materials described herein.

In the illustrated example, the base support 14 may be affixed directly to, and thus be in physical contact with, the complementary metal oxide semiconductor chip 46 through one or more securing mechanisms (e.g., adhesive, bond, fasteners, and the like). It is to be understood that the base support 14 may be removably coupled to the complementary metal oxide semiconductor (CMOS) chip 46.

The CMOS chip 46 includes a plurality of stacked layers 49 including, for example, silicon layer(s), dielectric layer(s), metal-dielectric layer(s), metal layer(s), etc.). The stacked layers 49 make up the device circuitry, which includes detection circuitry.

The CMOS chip 46 includes optical components, such as optical sensor(s) 50 and optical waveguide(s) 52. The optical components are arranged such that each optical sensor 50 at least substantially aligns with, and thus is operatively associated with, a single optical waveguide 52 and a single active area 32 of the flow cell 12C. However, in other examples, a single optical sensor 50 may receive photons through more than one optical waveguide 52 and/or from more than one active area 32.

The single optical sensor 50 may be a light sensor that includes one pixel or more than one pixel. As an example, each optical sensor 50 may have a detection area that is less than about 50 μm². As another example, the detection area may be less than about 10 μm². As still another example, the detection area may be less than about 2 μm². In the latter example, the optical sensor 50 may constitute a single pixel. An average read noise of each pixel the optical sensor 50 may be, for example, less than about 150 electrons. In other examples, the read noise may be less than about 5 electrons. The resolution of the optical sensor(s) 50 may be greater than about 0.5 megapixels (Mpixels). In other examples, the resolution may be greater than about 5 Mpixels, or greater than about 10 Mpixels.

Also as used herein, a single optical waveguide 52 may be a light guide including a cured filter material that i) filters the excitation light 54 (propagating from an exterior of the flow cell 12C into the flow channel 48), and ii) permits the light emissions (not shown, resulting from reactions at the active area) to propagate therethrough toward corresponding optical sensor(s) 50. In an example, the optical waveguide 52 may be, for example, an organic absorption filter. As a specific example, the organic absorption filter may filter excitation light 54 of about 532 nm wavelength and permit light emissions of about 570 nm or more wavelengths. The optical waveguide 52 may be formed by first forming a guide cavity in a dielectric layer 56, and then filling the guide cavity with a suitable filter material.

The optical waveguide 52 may be configured relative to the dielectric material 56 in order to form a light-guiding structure. For example, the optical waveguide 52 may have a refractive index of about 2.0 so that the light emissions are substantially reflected at an interface between the optical waveguide 52 and the surrounding dielectric material 56. In certain examples, the optical waveguide 52 is selected such that the optical density (OD) or absorbance of the excitation light 104 is at least about 4 OD. More specifically, the filter material may be selected and the optical waveguide 52 may be dimensioned to achieve at least 4 OD. In other examples, the optical waveguide 52 may be configured to achieve at least about 5 OD or at least about 6 OD.

As mentioned, in this example flow cell 12C, the base support 14 is made of the passivation material. At least a portion of the base support 14 is in contact with a first embedded metal layer 58 of the CMOS chip 46 and also with an input region 60 of the optical waveguide 52. The contact between the base support 14 and the first embedded metal layer 58 may be direct contact or may be indirect contact through a shield layer 62.

This example of the base support 14 (passivation material) may provide one level of corrosion protection for the embedded metal layer 58 of the CMOS chip 46 that is closest in proximity to the base support 14. As described herein, the passivation material is transparent to the light emissions resulting from reactions at the active region 32 (e.g., visible light), and is at least initially resistant to the fluidic environment and moisture that may be introduced into or present in the flow channel 48.

As shown in FIG. 5, each active area 32 of the flow cell 12C is at least substantially aligned with the input region 60 of a single optical waveguide 52. As such, light emissions at the active area 32 may be directed into the input region 60, through the waveguide 52, and to an associated optical sensor 50. In other examples, one active area 32 (e.g., a lane 20) may be aligned with several input regions 60 of several optical waveguides 50. In still other examples, several active area 32 (e.g., a subset of depression 22) may be aligned with one input region 60 of one optical waveguide 52.

The embedded metal layer 58 may be any suitable CMOS metal, such as aluminum (AI), aluminum chloride (AlCu), tungsten (W), nickel (Ni), or copper (Cu). The embedded metal layer 58 is a functioning part of the CMOS AVdd line, and through the stacked layers 49, is also electrically connected to the optical sensor 50. Thus, the embedded metal layer 58 participates in the detection/sensing operation.

It is to be understood that the other optical sensors 50 and associated components may be configured in an identical or similar manner. It is also to be understood, however, that the CMOS chip 46 may not be manufactured identically or uniformly throughout. Instead, one or more optical sensor 50 and/or associated components may be manufactured differently or have different relationships with respect to one another.

The stacked layers 49 may include interconnected conductive elements (e.g., conductors, traces, vias, interconnects, etc.) that can conduct electrical current. The circuitry may be configured for selectively transmitting data signals that are based on detected photons. The circuitry may also be configured for signal amplification, digitization, storage, and/or processing. The circuitry may collect and analyze the detected light emissions and generate data signals for communicating detection data to a bioassay system. The circuitry may also perform additional analog and/or digital signal processing in the CMOS chip 46.

The CMOS chip 46 may be manufactured using integrated circuit manufacturing processes. The CMOS chip 46 may include multiple layers, including a sensor base/layer (e.g., a silicon layer or wafer). The sensor base may include the optical sensor 50. When the CMOS chip 46 is fully formed, the optical sensor 50 may be electrically coupled to the rest of the circuitry in the stack layer 48 through gate(s), transistor(s), etc.

As used in reference to FIG. 5, the term “layer” is not limited to a single continuous body of material unless otherwise noted. For example, the sensor base/layer may include multiple sub-layers that are different materials and/or may include coatings, adhesives, and the like. Furthermore, one or more of the layers (or sub-layers) may be modified (e.g., etched, deposited with material, etc.) to provide the features described herein.

The stacked layers 49 include a plurality of metal-dielectric layers. Each of these layers includes metallic elements (e.g., M1-M5, which may be, for example, W (tungsten), Cu (copper), Al (aluminum), or any other suitable CMOS conductive material) and dielectric material 56 (e.g., SiO₂). Various metallic elements M1-M5 and dielectric materials 56 may be used, such as those suitable for integrated circuit manufacturing.

In the example shown in FIG. 5, each of the plurality of metal-dielectric layers L1-L6 includes both metallic elements M1, M2, M3, M4, M5 and dielectric material 56. In each of the layers L1-L6, the metallic elements M1, M2, M3, M4, M5 are interconnected and are embedded within dielectric material 56. In some of the metal-dielectric layers L1-L6, additional metallic elements may also be included. Some of these additional metallic elements may be used to address individual pixels through a row and column selector. The voltages at these elements may vary and switch between about-1.4 V and about 4.4 V depending upon which pixel the device is reading out.

The configuration of the metallic elements M1, M2, M3, M4, M5 and dielectric layer 56 in FIG. 5 is illustrative of the circuitry, and it is to be understood that other examples may include fewer or additional layers and/or may have different configurations of the metallic elements M1-M5.

In the example shown in FIG. 5, the shield layer 62 is in contact with at least a portion of the base support 14. The shield layer 62 has an aperture at least partially adjacent to the input region 60 of the optical waveguide 52. This aperture enables the active area 32 (and at least some of the light emissions therefrom) to be optically connected to the waveguide 52. It is to be understood that the shield layer 62 may have an aperture at least partially adjacent to the input region 60 of each optical waveguide 52. The shield layer 62 may extend continuously between apertures (input regions 60).

The shield layer 62 may include any material that can block, reflect, and/or significantly attenuate the light signals that are propagating through the flow channel 48. The light signals may be the excitation light 54 and/or the light emissions from the active area(s) 32. As an example, the shield layer 62 may be tungsten (W).

It is to be understood that the flow cell 12C shown in FIG. 5 may also be used for optical detection.

While not shown in FIG. 5, it is to be understood that the solid polymer matrix 42′ generated to graft the primers 30A, 30B to the lane(s) 20 and/or depression(s) 22 may remain in the lane(s) 20 and/or depression(s) 22 while the patterned substrate 10A, 10B, 10C is bonded to the lid 44 and while the flow cell 12C is integrated with the CMOS chip 46. It may be desirable for the solid polymer matrix 42′ to remain in the lane(s) 20 and/or depression(s) 22 during bonding to protect the polymer 28 and the attached primers 30A, 30B. In this example, the solid polymer matrices 42′ may be removed (as described herein) from the patterned structures 10A, 10B, 10C of the flow cell 12C before a sequencing operation is performed.

Alternatively, the solid polymer matrix 42′ generated to graft the primers 30A, 30B to the lane(s) 20 and/or depression(s) 22 may be removed from the lane(s) 20 and/or depression(s) 22 before the patterned substrates 10A, 10B, 10C is bonded to the lid 44. In this example, the respective patterned substrate 10A, 10B, 10C would be exposed to a suitable removal solvent (e.g., water) and rinsed before bonding takes place.

### Flow Cell Protective Coating

When the solid polymer matrix 42′ is removed before bonding is performed to generate an example of the flow cell 12A, 12B, 12C, a protective coating (not shown) may be generated in the flow channel 48 by introducing a protective coating solution to the flow cell 12A, 12B, 12C; and drying the protective coating solution to form a solid protective coating. The protective coating solution may be any example of the grafting solution 40 set forth herein that does not include the primers 30A, 30B. This solid protective coating may be desirable when the flow cell 12A, 12B, 12C is to be shipped and/or stored prior to usage. This solid protective coating may be removed, e.g., using water, when it is desirable to use the flow cell 12A, 12B, 12C in a sequencing operation.

### Sequencing Methods

For sequencing using the patterned structures 10A, 10B, 10C or the flow cells 12A, 12B, 12C disclosed herein, DNA sample strands (i.e., library templates) may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 30A, 30B. In some examples, the fragments from a single nucleic acid sample have the same adapters added thereto. The final library templates include the DNA or cDNA fragment and adapters at both ends. The DNA or cDNA fragment represents the portion of the final DNA sample strand that is to be sequenced.

Amplification of the final library templates is formed on the open patterned structure 10A, 10B, 10C or in the flow cell 12A, 12B, 12C. The final library templates are added to the patterned structure 10A, 10B, 10C or the flow cell 12A, 12B, 12C in an amplification mix, which includes a liquid carrier and a high-fidelity DNA polymerase. The liquid carrier may include a buffer (e.g., a Tris-HCl buffer or 0.5× saline sodium citrate (SSC) buffer), acetic acid, acetone, acetonitrile, benzene, butanol, diethylene glycol, diethyl ether, dimethyl formamide, ethanol, glycerin, methane, pyridine, triethyl amine, etc. Surfactants/dispersants, such as sodium dodecyl sulfate (SDS), (CTAB) may also be included.

During amplification, one or more library templates is/are hybridized, for example, to the primers 30A, 30B in the lane(s) 20 and/or in the depressions 22. Amplification is initiated to form clusters of the template strands across the lane(s) 20 and/or in the depressions 22.

In one example, amplification involves cluster generating. In one example of cluster generation, the seeded library template is copied from the hybridized primers 30A, 30B by 3′ extension using the high-fidelity DNA polymerase. The original library template is denatured, leaving the copies (amplicons) immobilized in the lane(s) 20 or the depressions 22. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters in the lane(s) 20 and/or the depressions 22. This example of clustering is referred to as bridge amplification, and is one example of the amplification that may be performed. It is to be understood that other amplification techniques may be used.

As described herein, the primers 30A, 30B have orthogonal cleaving chemistry. A chemical agent or an enzymatic cleaving agent may be introduced to remove either the forward amplicons or the reverse amplicons generated during amplification. After cleavage, the other of the reverse amplicons or the forward amplicons remain in the lane(s) 20 and/or the depressions 22.

Sequencing primers may then be introduced to the open patterned structure 10A, 10B, 10C or the flow cell 12A, 12B, 12C. The sequencing primers hybridize to a complementary portion of the sequence of the remaining reverse amplicons or forward amplicons. These sequencing primers render the amplicons ready for sequencing.

An incorporation mix including labeled nucleotides may then be introduced to the open patterned structure 10A, 10B, 10C or in the flow cell 12A, 12B, 12C. In addition to the labeled nucleotides, the incorporation mix may include water and/or an ionic salt buffer fluid, such as saline citrate at milli-molar to molar concentrations, sodium chloride, potassium chloride, phosphate buffered saline, etc., and/or other buffers, such as tris(hydroxymethyl)aminomethane (TRIS) or (4-(2-hydroxyethyl)-1-piperazineethanesulfonic acid) (HEPES). The liquid carrier may also include catalytic metal(s) intended for the incorporation reaction, such as Mg²⁺, Mn²⁺, Ca²⁺, etc. A single catalytic metal or a combination of catalytic metals may be used, and the total concentration may range from about 0.01 mM to about 100 mM. The incorporation mix also includes a polymerase that can accept the labeled nucleotides, and that can successfully incorporate the nucleotide base into a nascent strand along an amplicon. Examples polymerases include those polymerases from family A, such as Bsu Polymerase, Bst Polymerase, Taq Polymerase, T7 Polymerase, and many others; polymerases from families B and B2, such as Phi29 polymerase and other highly processive polymerases (family B2), Pfu Polymerase (family B), KOD Polymerase (family B), 90N (family B), and many others; polymerases from family C, such as Escherichia coli DNA Pol III, and many others, polymerases from family D, such as Pyrococcus furiosus DNA Pol II, and many others; polymerases from family X, such as DNA Pol μ, DNA Pol β, DNA Pol σ, and many others.

When the incorporation mix is introduced to the open patterned structure 10A, 10B, 10C or into the flow cell 12A, 12B, 12C, the mix contacts the remaining amplicons. The incorporation mix is allowed to incubate, and labeled nucleotides (including optical labels) are incorporated by respective polymerases into the nascent strands along the amplicons.

During incorporation, one of the labeled nucleotides is incorporated, by a respective polymerase, into one nascent strand that extends one sequencing primer and that is complementary to one of the amplicons. Incorporation is performed in a template strand dependent fashion, and thus detection of the order and type of labeled nucleotides added to the nascent strand can be used to determine the sequence of the amplicon and thus the original DNA sample stands. Incorporation occurs in at least some of the amplicons across the lane(s) 20 and/or depressions 22 during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the patterned structure 10A, 10B, 10C or flow cell 12A, 12B, 12C during a wash cycle.

Without further incorporation taking place, the most recently incorporated labeled nucleotides can be detected through an imaging event or a data collection event.

During the imaging event, an illumination system may provide excitation light to the patterned structure 10A, 10B, 10C or flow cell 12A, 12B, 12C. The optical labels of the incorporated labeled nucleotides emit optical signals in response to the excitation light. An optical imager captures images of the optical signals.

During the data collection event, the illumination system may provide an excitation light (e.g., light 54 in FIG. 5) to the flow cell 12C. Like the imaging event, the optical labels of the incorporated labeled nucleotides emit optical signals in response to the excitation light. The light emissions (e.g., photons) are directed through the waveguide 52 toward the optical sensor 50. The circuitry of the CMOS chip 46 collects and analyzes the detected light emissions and generates data signals. The circuitry can communicate/transmit the data signals to a bioassay system. The circuitry may also be configured for signal amplification, digitization, storage, and/or processing. The circuitry may also perform additional analog and/or digital signal processing.

After imaging or data collection is performed, a cleavage mix may then be introduced to the patterned structure 10A, 10B, 10C or flow cell 12A, 12B, 12C. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the amplicons are sequenced. The nascent strands may be dehybridized and then the processes of cluster generation and sequencing may be repeated for whichever of the forward or reverse amplicons has not been sequenced.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A method, comprising:
using a liquid-phase thin-film deposition technique, applying a grafting solution to a patterned substrate, wherein:
the patterned substrate includes:
a lane surrounded by interstitial regions or a plurality of depressions separated by interstitial regions; and
a polymer in the lane or in each of the plurality of depressions, the polymer being functionalized with a first click reaction moiety; and
the grafting solution includes:
a solvent;
a polymer matrix material dissolved in the solvent; and
primers of a primer set dissolved in the solvent, each of the primers being terminated with a second click reaction moiety; and
drying the grafting solution, thereby forming a solid polymer matrix and attaching at least some of the primers to the polymer i) via the first and second click reaction moieties and ii) in at least a portion of the lane or in at least some of the plurality of depressions.

2. The method as defined in claim 1, further comprising removing the solid polymer matrix and unattached primers by rinsing the patterned substrate with a solvent of the solid polymer matrix, whereby the attached primers remain in the at least the portion of the lane or the at least some of the plurality of depressions.

3. The method as defined in claim 2, further comprising:
introducing a protective coating solution to the patterned substrate; and
drying the protective coating solution to form a solid protective coating.

4. The method as defined in claim 3, wherein prior to introducing the protecting coating solution or after drying the protective coating solution, the method further comprises bonding the patterned substrate to a second patterned substrate or to a lid to form a flow cell.

5. The method as defined in claim 1, wherein while the solid polymer matrix and unattached primers are present in the at least the portion of the lane or in the at least some of the plurality of depressions, the method further comprises bonding the patterned substrate to a second patterned substrate or to a lid to form a flow cell.

6. The method as defined in claim 1, wherein the grafting solution includes:
up to 15% (mass to volume) of the polymer matrix material; and
up to 10% (mass to volume) of the primers.

7. The method as defined in claim 1, wherein the polymer matrix material is selected from the group consisting of a water-soluble non-cationic synthetic polymer; a water-soluble natural polysaccharide or a derivative thereof; a water-soluble natural protein or a derivative thereof; and combinations thereof.

8. The method as defined in claim 1, wherein the grafting solution further includes an additive selected from the group consisting of a sugar, a surfactant, and combinations thereof.

9. The method as defined in claim 8, wherein:
the grafting solution includes:
up to 15% (mass to volume) of the polymer matrix material;
up to 35% (mass to volume) of the additive; and
up to 10% (mass to volume) of the primers; and
a total (mass to volume) of the polymer matrix material and the additive ranges from about 5% to about 40%.

10. The method as defined in claim 9, wherein:
the polymer matrix material includes a water-soluble non-cationic synthetic polymer;
the water-soluble non-cationic synthetic polymer is a polyvinyl alcohol/polyethylene glycol graft copolymer;
the additive is the sugar; and
the sugar is sucrose.

11. The method as defined in claim 1, wherein the liquid-phase thin-film deposition technique is selected from the group consisting of dispense coating, slot-die coating, and inkjet printing.

12. The method as defined in claim 1, wherein the first click reaction moiety is azide and the second click reaction moiety is bicyclononyne.

13. The method as defined in claim 1, wherein the first click reaction moiety is azide and the second click reaction moiety is dibenzocyclooctyne.

14. The method as defined in claim 1, wherein the first click reaction moiety is tetrazine and the second click reaction moiety is trans-cyclooctene.

15. The method as defined in claim 1, wherein drying the grafting solution is accomplished at ambient temperature.

16. The method as defined in claim 1, wherein drying the grafting solution involves exposing the applied grafting solution to heat ranging from about 25° C. to about 120° C.

17. The method as defined in claim 1, further comprising allowing the solid polymer matrix to remain in the lane or the plurality of depressions during shipping, storage, or combinations thereof.

18. The method as defined in claim 1, wherein:
the patterned substrate is part of a flow cell including:
the patterned substrate bonded to a lid or a second patterned substrate; and
a flow channel defined between the patterned substrate and the lid or the second patterned substrate;
the liquid-phase thin-film deposition technique is a flow through technique; and
drying the grafting solution involves introducing an air or nitrogen gas bubble into the flow cell while the grafting solution is present in the flow channel.

19. A method, comprising:
using a liquid-phase thin-film deposition technique, applying a grafting solution to a patterned substrate, wherein:
the patterned substrate includes:
a lane surrounded by interstitial regions or a plurality of depressions separated by interstitial regions; and
a polymer in the lane or in each of the plurality of depressions, the polymer being functionalized with a first click reaction moiety; and
the grafting solution includes:
a solvent;
a polymer matrix material dissolved in the solvent; and
a plurality of biomolecules dissolved in the solvent, each of the plurality of the biomolecules being terminated with a second click reaction moiety; and
drying the grafting solution, thereby forming a solid polymer matrix and attaching at least some of the plurality of biomolecules to the polymer i) via the first and second click reaction moieties and ii) in at least a portion of the lane or in at least some of the plurality of depressions.

20. The method as defined in claim 19, wherein the plurality of biomolecules is selected from the group consisting of biotin, a protein, an enzyme, DNA, RNA, an aptamer, or a combination of a primer set and a quality control oligonucleotide.
